(19) United States
(12) Patent Application Publication
(10) Pub. No.: US 2022/0032122 A1
(43) Pub. Date: Feb. 3, 2022

(54) METHOD FOR MANAGING REMOTE DATA

(71) Applicant: Michael James Gearon, Victoria (AU)

(72) Inventor: Michael James Gearon, Victoria (AU)

(21) Appl. No.: 17/389,877

(22) Filed: Jul. 30, 2021

(30) Foreign Application Priority Data
Jul. 30, 2020 (AU) 2020902679

(51) Int. Cl. A63B 24/00; H04Q 9/04; A63B 21/22; G16H 20/30; G16H 40/67

(52) U.S. Cl. CPC A63B 24/0062; H04Q 9/04; A63B 21/225; G16H 20/30; A63B 2220/62; H04Q 2209/845; A63B 2220/51; A63B 2220/20; G16H 40/67

(57) ABSTRACT

The present disclosure provides a method of managing data, remotely generated, by, for example, resistance-based fitness devices, in which variable RPM measurement from a flywheel or similar mechanism is replaced with standardized digitally generated RPM measurement. The resulting data packet can be stored with other packets from the same event as a record. Stored records may be accurately compared with other such records from the same or other users for a variety of purposes. A computer-readable medium storing instructions for executing the method is also provided.

### FIELD OF THE DISCLOSURE

The disclosure relates to management of remotely generated data. Particularly, to a method of providing synchronization and normalization of data generated remotely by resistance-based fitness devices.

### BACKGROUND OF THE DISCLOSURE

Personal fitness devices, for use in the home or other isolated locations, have been popular for decades, providing users with the physical and mental benefits of actual outdoor exercise with none of the inconvenience, location, and scheduling demands thereof.

Events in recent years compelling increasing numbers of people to limit or even cease altogether excursions into public environments have resulted in skyrocketing popularity of such devices.

As technology evolves, manufacturers continue to apply such developments to a wide range of these devices, in many cases to enhance satisfaction by more completely replicating actual real-world exercise experience for the user.

One such development, often applied to resistance-based devices, utilizes internet capability to enable a central platform to accept generated use data from individual participants. Received data can be managed and collated with other users' data to construct cooperative or competitive scenarios shared among all involved users, for a heightened replication of actual recreation for participants.

The concerned devices, such as rowing machines, often employ a functioning flywheel arrangement to sustain generated force between applications of work by the user. In the case of rowing machines, the force is generated by rowing strokes. The flywheel is driven to spin by the applied energy of the stroke. Once the actual stroke is complete, however, the flywheel continues to spin. The force provided thereby maintains some of the originally generated energy whilst the user returns to the starting position to begin another stroke. The maintained energy from the flywheel prevents the need for the user to overcome complete inertia when beginning the new stroke. Of course, the flywheel begins to slow once each stroke is complete, but some measure of momentum, albeit decreasing, is still provided. The same operating principle can be applied to devices providing cycling, skiing, and other forms of exercise.

A major impediment to the previously described central platform for gathering and collating exercise data, however, lies in the fact that among devices generating gathered work data, performance of the incorporated flywheel elements is completely non-standard. Each flywheel's operation, that is, the rate at which it spins up with application of force and slows down once no force is applied, is essentially unique, based on construction, deployment, various environmental factors, and other conditions. This prevents the platform from objectively measuring the virtual distance covered by the user and the speed at which it is covered. The result is that comparison or matching of user performance is largely inaccurate.

Accordingly, what is called for is a means of synchronizing and normalizing data generated remotely by resistance-based fitness devices.

### SUMMARY OF THE DISCLOSURE

The present disclosure provides a method of managing remotely generated data. Configured data received comprises at least one modular measurement of force, distance, and time (FDT) created by a discrete physical effort, and at least one RPM measurement of a flywheel or similar mechanism storing rotational energy. The at least one RPM measurement is associated directly with at least one of the modular measurements and is split between progressively increasing RPMs and progressively decreasing RPMs.

The method replaces each remotely generated RPM measurement with a digitally generated RPM measurement. The digitally generated RPM measurement is configured according to the at least one remotely generated FDT modular measurement with which the replaced remotely generated RPM measurement is associated. The digitally generated RPM measurement is, further, based on a specific algorithm applying characteristics of an actual physical flywheel and comprises a set of progressively increasing RPMs and progressively decreasing RPMs, with the digitally generated RPM measurement affected by the same applied physical conditions.

The newly created packet, comprising the at least one FDT modular measurement and the corresponding at least one standardized digitally generated RPM measurement, is then stored. Multiple packets from a single event can be stored together to collectively constitute a record of cumulative physical effort expended in the event.

The method may further analyze the now standardized records of cumulative physical effort expended in an event alongside other such records, generated by the same or other users employing the same type of device, where the analysis is made for purposes of competition, relative improvement in performance, comparison with established records, or other form of relative assessment. Such comparisons are considerably more accurate than with the originally generated data.

The remotely generated data as received can be generated by use of a resistance-based exercise device, such as a rowing machine, exercise bicycle, ski exerciser, or other means.

Those skilled in the art will recognize or be able to ascertain, using no more than routine experimentation, many equivalents to the specific embodiments described herein. The scope of the present embodiments described herein is not intended to be limited to the provided description, but rather is as set forth in the appended claims. Those of ordinary skill in the art will appreciate that various changes and modifications to this description may be made without departing from the spirit or scope of the present invention, as defined in the following claims.

What is claimed is:

1. A method of managing remotely generated data, the data comprising at least one modular measurement of force, distance, and time (FDT) created by discrete physical effort, and at least one RPM measurement of a flywheel or similar mechanism storing rotational energy, the at least one RPM measurement associated directly with at least one of the modular measurements and split between progressively increasing RPMs and progressively decreasing RPMs, the method comprising;
replacing each of the remotely generated RPM measurements with a digitally generated RPM measurement configured according to the at least one remotely generated FDT modular measurement with which the replaced remotely generated RPM measurement is associated, wherein the digitally generated RPM measurement is based on a specific algorithm applying characteristics of an actual physical flywheel and comprises a set of progressively increasing RPMs and progressively decreasing RPMs, and wherein every digitally generated RPM measurement is affected by the same applied physical conditions; and
storing the newly created packet comprising the at least one FDT modular measurement and the corresponding at least one digitally generated RPM measurement, wherein multiple packets from a single event can be stored together to collectively constitute a record of cumulative physical effort expended in the event.

2. The method of claim 1, in which the remotely generated data is generated by use of a resistance-based exercise device, such as a rowing machine, exercise bicycle, ski exerciser, or other.

3. The method of claim 2, in which records of cumulative physical effort expended in an event may be analyzed alongside other such records generated by the same or other users employing the same type of device, wherein the analysis is made on the basis of competition, relative improvement in performance, comparison with established records, or other form of relative assessment.

4. A computer-readable medium storing instructions that, when executed by a computer, cause it to perform a method of managing remotely generated data, the data comprising at least one modular measurement of force, distance, and time (FDT) created by discrete physical effort, and at least one RPM measurement of a flywheel or similar mechanism storing rotational energy, the at least one RPM measurement associated directly with at least one of the modular measurements and split between progressively increasing RPMs and progressively decreasing RPMs, the method comprising;
replacing each of the remotely generated RPM measurements with a digitally generated RPM measurement configured according to the at least one remotely generated FDT modular measurement with which the replaced remotely generated RPM measurement is associated, wherein the digitally generated RPM measurement is based on a specific algorithm applying characteristics of an actual physical flywheel and comprises a set of progressively increasing RPMs and progressively decreasing RPMs, and wherein every digitally generated RPM measurement is affected by the same applied physical conditions; and
storing the newly created packet comprising the at least one FDT modular measurement and the corresponding at least one digitally generated RPM measurement, wherein multiple packets from a single event can be stored together to collectively constitute a record of cumulative physical effort expended in the event.

5. The computer-readable medium of claim 4, in which the remotely generated data is generated by use of a resistance-based exercise device, such as a rowing machine, exercise bicycle, ski exerciser, or other.

6. The computer-readable medium of claim 5, in which records of cumulative physical effort expended in an event may be analyzed alongside other such records generated by the same or other users employing the same type of device, wherein the analysis is made on the basis of competition, relative improvement in performance, comparison with established records, or other form of relative assessment.
